(19) United States
(12) Patent Application Publication
(10) Pub. No.: US 2003/0229004 A1
(43) Pub. Date: Dec. 11, 2003

(54) Modulation of tumor cells using BER inhibitors in combination with a sensitizing agent and DSBR inhibitors

(75) Inventors: David A. Zarling, Menlo Park, CA (US); Gurucharan Reddy, Fremont, CA (US); Pietro Taverna, Fremont, CA (US)

Correspondence Address: BOZICEVIC, FIELD & FRANCIS LLP, 200 MIDDLEFIELD RD, SUITE 200, MENLO PARK, CA, 94025, US

(73) Assignee: Pangene Corporation

(21) Appl. No.: 10/394,431

(22) Filed: Mar. 20, 2003

Related U.S. Application Data
(60) Provisional application No. 60/367,447, filed on Mar. 20, 2002.
(60) Provisional application No. 60/448,732, filed on Feb. 21, 2003.

(51) Int. Cl. A61K 48/00; A61K 38/17; A61K 31/00

(52) U.S. Cl. 514/1; 514/2; 514/44

(57) ABSTRACT

Methods and compositions are providing for modulating cellular activity. In the subject methods, target cells are contacted with both a BER inhibitor and a sensitizing agent, e.g., either a radiosensitizing agent and/or a chemotherapeutic agent, where the cells may optionally be contacted with a DSBR inhibitor, such as a RAD inhibitor, e.g., a RAD51 inhibitor. Also provided are pharmaceutical preparations, as well as kits thereof, that find use in practicing the subject methods. The subject methods find use in a variety of different applications, including the treatment of hosts suffering from cellular proliferative

### FIELD OF THE INVENTION

The invention relates to methods and compositions for inhibiting the proliferation of cells and sensitizing cells to radiation therapy and DNA damaging chemotherapeutics, and in particular, treating cancer cells and individuals in vivo, including intra-operative treatments, by administration of a combination of DNA chemo-or-radio-sensitizing drugs, BER (DNA Base Excision Repair) pathway inhibitors and DSBR (DNA Double Strand Break Repair) pathway inhibitors.

### BACKGROUND OF THE INVENTION

Many valuable and life-saving chemotherapeutic drugs, actively used in the clinic, achieve their effect by damaging DNA in proliferating cells. Examples are 1) alkylating agents, such as temozolomide, sarmustine, chlorambucil, melphalan, dacarbazine, BCNU and SCNU. 2) nucleoside analogues, such as fludarabine, iodouridinedeoxyribose, gemcitabine, and fluorodeoxyuridine, and 3) radiation therapy. All of these treatments result in cytotoxic modifications in DNA bases, which lead to Single Strand Breaks (SSB) in the drug-incorporated DNA strand as well as in the un-substituted complementary-strand DNA. These SSBs subsequently result in increase in the amount of Double Strand Breaks (DSB) (Fornace, Dobson et al. 1990) which, if not repaired properly, result in cell death (Kinsella, Dobson et al. 1987). In addition, in cases when cells are resistant to DNA damaging agents, different radio- and chemo-sensitizing agents have been used to increase the sensitivity to DNA damaging radiation and chemotherapeutics.

Since DNA damage is potentially lethal for cells, practically every living cell has the potential to repair certain damages to its DNA. Two of the major pathways for DNA repair are the Base Excision Repair (BER) pathway and the double strand break repair (DSBR) pathway. BER is the major pathway responsible for repairing single-strand breaks caused by base modifications in DNA, including those generated by the clinically used anticancer agents, while DSBR is responsible for repair of lethal DSBs.

Often proliferatingtumor or viral infected cells are resistant to chemo- and radiotherapy due to over-expression of the DNA repair mechanisms. Since SSBs can be converted to DSBs, even if one of these pathways is blocked, the other pathway may enable cells to repair damage and sustain viability. Agents that inhibit BER and DSBR in a specific and potent manner sensitize proliferating cells to a broad spectrum of anticancer agents. Since cancer cells rely on DNA repair to allow them to grow rapidly, this sensitization would enhance the specificity of cancer therapy and allow more effective therapy with lower side effects than is possible with current therapeutic regimens.

The present invention, for the first time, provides methods and compositions to inhibit cell proliferation, comprising administration of both BER pathway and DSBR pathway inhibitors, combined with DNA chemo-or-radio-sensitizing drugs. The invention further provides both BER pathway and DSBR pathway inhibitor molecules that disrupt mammalian single and double stranded break repair. Moreover, the invention provides methods to treat diseased cells or individuals by administering a composition comprising BER pathway and DSBR pathway inhibitors. Additionally, the invention provides methods of inducing sensitization to radiation, aklylating agents and other DNA damaging chemotherapeutics in vivo using BER pathway and DSBR pathway inhibitors. Other aspects of the invention are described below.

### RELATED ART

A. DNA Single Strand Break Repair

DNA-SSB (single strand breaks) are one of the most frequent lesions occurring in cellular DNA either spontaneously or as intermediates of enzymatic repair of base damage during Base Excision Repair (BER) (Lindahl 1993; Caldecott 2001). In this repair pathway, which follows the removal of a damaged base by a DNA glycosylase, the resulting apurinic/apyrimidinic (AP) site can be processed by (1) AP endonuclease (Ape1) cleavage leaving a 5′ deoxyribose-phosphate (2) by an AP lyase activity leaving a 3′ β-elimination product. The subsequent removal of these AP sites by DNA Polymerase β, or by a PCNA-dependent polymerase, allows the repair synthesis to fill-in either a single nucleotide (for Pol β) or a longer repair patch (for Pol δ/ε), which are then re-ligated (Wilson 1998). If SSB sites, arising as repair intermediates, are not promptly and efficiently processed, clusters of damaged sites and stalled replication forks will form, resulting in the formation of DSBs with lethal consequences for the cell (Chaudhry and Weinfeld 1997; Harrison, Hatahet et al. 1998).

B. BER Pathway Protein AP Endonuclease (Ape1)

The second enzyme in the human DNA BER pathway, Ape1, contributes to the repair of DNA damage by hydrolyzing the phosphodiester backbone immediately 5′ to an abasic (AP) site. Ape1 is a 37 kDa protein with an N-terminal domain, which contains the nuclear localization signal and a region required for a redox function, and a C-terminal region containing the endonuclease activity. Ape1 is a multifunctional protein that is not only responsible for repair of AP sites, but also functions as a redox factor maintaining transcription factors in an active, reduced state. Ape1 has been shown to stimulate the DNA binding activity of numerous transcription factors that are involved in cancer promotion and progression such as Fos, Jun, NFkB, PAX, HIF-1a, HLF and p53 and has been shown to interact with Ku70/80 which is involved in double strand break repair. Bacteria, yeast or human cells lacking AP endonuclease repair activity are hypersensitive to agents (e.g. alkylating or oxidizing) that induce the formation of AP sites (Demple and Harrison 1994). Moreover, targeted reduction of APE1 protein by specific anti-sense oligonucleotides renders mammalian cells hypersensitive to MMS, H₂O₂, and bleomycin (Ono, Furuta et al. 1994; Walker, Craig et al. 1994; Herring, West et al. 1998).

C. BER Pathway Inhibitor Methoxyamine

Methoxyamine (MX) is an alkoxyamine derivative able to block the single nucleotide BER pathway by a reaction with the aldehydic C1 atom of the acyclic sugar left in the DNA abasic AP site following the glycosylase-driven removal of the damaged nucleotide. The MX-adducted AP site is a stable intermediate, refractory to the dRPase lyase activity of Polymerase β and to the AP endonuclease cleavage. Chemical inhibition of BER by MX is a valid pharmacological strategy to overcome resistance to the methylating chemotherapeutic agent temozolomide (Liu, Taverna et al. 1999; Taverna, Liu et al. 2001; Liu, Nakatsuru et al. 2002). Tomicic et al. (Tomicic, Thust et al. 2001) reported that MX sensitized wild type and Polβ-complemented mouse fibroblasts to the cytotoxicity of Ganciclovir, a nucleoside analogue used as an antiviral agent and used in experimental suicide gene therapy following transduction of tumor cells with the HSVtk gene. More recently, MX-mediated modulation of Base Excision Repair was shown to affect cell sensitivity to hydrogen peroxide (H₂O₂) (Horton, Baker et al. 2002) and to UVA1 radiation (Kim, Chakrabarty et al. 2002).

D. Halogenated Nucleotide Analogues

Dillehay et al. first suggested a possible role for BER in the cytotoxicity of halogenated thymidine analogues (Dillehay, Thompson et al. 1984). More recently, BER-mediated 5-Chloro-2′-deoxyuridine (CldUrd) cytotoxicity was believed to result from the removal of uracil incorporated in DNA secondary to the inhibition of thymidylate synthase (TS) by CldUMP, one of the metabolic intermediates of CldUrd (Brandon, Mi et al. 2000). Several other studies have also described mismatch-specific enzymes including Thymine DNA Glycosylase (TDG) and Methyl-CpG Binding Endonuclease 1 (MED1, also known as MBD4), which removes uracil, 5-bromouracil and 5-fluorouracil residues from DNA (Neddermann and Jiricny 1994; Petronzelli, Riccio et al. 2000).

Modified nucleosides used in anticancer and antiviral therapies include the 5′-substituted halogenated pyrimidine analogues Iododeoxyuridine (IUDR) and Bromodeoxyuridine (BUdR) (Kinsella 1996), DNA replication inhibiting nucleosides such as Fludarabine (FaraA) (Keating, Kantarjian et al. 1989), Cytarabine (araC) (Keating, Estey et al. 1985) and Gemcitabine (Gemzar) (Stomiolo, Enas et al. 1999) or pyrimidinone nucleosides like 5-iodo-2′-deoxyribose (IPdR) (Kinsella, Vielhuber et al. 2000). These modified nucleosides have been studied for several years as potential cancer chemotherapeutic and chemo-or-radiosensitizing agents and more recently their clinical use has produced favorable results against a broad spectrum of tumors. However, the precise molecular mechanisms by which these nucleoside analogs produce cytotoxicity in mammalian cells are not fully understood. Based on cellular and biochemical studies, the extent of incorporation of nucleoside analogues into DNA has been consistently shown to be linearly correlated with the extent of radiosensitization and cytotoxicity in normal and malignant cells (Miller, Fowler et al. 1992).

Incorporation of halogenated pyrimidine analogues results in an increased amount of initial DNA damage following Ionizing Radiation (IR) as measured by an increase in DNA Single Strand Breaks (SSB) and Double Strand Breaks (DSB). Additionally, these analogues can affect the rate/extent of IR-damage repair. Based on these observations, the proposed biochemical mechanism of radio-sensitization is that the incorporated halogenated deoxyuridine reacts with radiation-induced hydrated electrons resulting in highly reactive uracilyl radicals and halide ions. DNA SSB are then produced by these reactive species in the drug-incorporated DNA strand as well as in un-substituted complementary-strand DNA which can then result in increased DSB (Kinsella, Dobson et al. 1987; Fornace, Dobson et al. 1990). Un-repaired or mis-repaired DNA DSBs finally result in cell death.

E. IodoUridineDeoxyRibose (IUDR)

The in vivo use of radiosensitizing pharmaceutical drugs poses major difficulties in cancer radiotherapy. IUDR (IodoUridineDeoxyRibose), a halogenated thymidine analogue, is a well characterized anti-herpes drug which is FDA approved. It can also be used as a radiosensitizer for human cancers, but is not approved for use due to a requirement for long intravenous infusions, which results in toxicity.

IUDR cytotoxicity and radiosensitization result, in part, from induction of DNA Single Strand Breaks (SSB) with subsequent enhanced DNA Double Strand Breaks (DSB) leading to cell death. We have published evidence that the increased IUDR cytotoxicity observed in cells lacking functional single-nucleotide BER can be explained by the increased number of DNA breaks left unrepaired following the removal of the iodouracil base from the DNA backbone (Taverna, Hwang et al. 2003). The presence of these DNA breaks may be explained by the recently proposed model of Wilstermann and Osheroff (Wilstermann and Osheroff 2001) wherein abasic sites left unrepaired within a Topoisomerase II DNA cleavage site act as Topo II poisons and significantly increase the enzyme-mediated DNA cleavage. These transient DNA breaks are converted to unrepaired double-strand breaks and, therefore, cause cell death.

F. IodoPyrimidinoneDeoxyRibose (IPDR)

IPDR is a well-characterized oral prodrug that is converted to IUDR by aldehyde oxidase in vivo. IPDR has been studied in animals, but has not been tested yet in human studies. The use of p.o. administered IPDR (5-iodo-2-pyrimidinone-2′-deoxyribose) as a prodrug for IUDR-mediated tumor radiosensitization is an approach under development by the group of Dr Kinsella (Kinsella, Schupp et al. 2000; Kinsella, Vielhuber et al. 2000). An aldehyde oxidase, most concentrated in rodent and human liver, efficiently converts IPDR to IUDR (Chang, Doong et al. 1992). An improved therapeutic gain for in vivo human tumor xenograft radiosensitization has been described using daily p.o. dosing of IPDR for 6 or 14 days compared to either p.o. or continuous infusion of IUDR for similar time periods. These treatments result in no significant systemic toxicity in nude mice and are associated with significant radiosensitization using human colon and brain cancer xenograft models. IUDR-related cytotoxicity and/or radiosensitization are directly correlated with the extent of IUDR-DNA incorporation replacing thymidine.

G. Double Strand Break Repair

In human cells, recombinational repair of DNA double strand breaks (DSBR) occurs either by homologous recombination (HR) or by non-homologous recombination (ie. non-homologous end-joining/NHEJ) (Modesti and Kanaar 2001; Pierce, Stark et al. 2001). Homologous recombination involves the Rad51, Rad52, Rad54, Rad55-57 and Rpa proteins. More recently, the Brca1 and Brca2 cancer-susceptibility proteins have been suggested to play a role in homologous DSBR through interactions with Rad50 and Rad51 respectively (Chen, Silver et al. 1999; Bhattacharyya, Ear et al. 2000; Bogliolo, Taylor et al. 2000). Brca1 may also mediate microhomology-mediated DNA end-joining, utilizing short base pair stretches at the DNA ends. Current models suggest that Rad51 is a stably-associated core component of the multi-protein HR-repair complex at sites of DNA damage and that its associated proteins, Rad52 and Rad54, rapidly and reversibly interact with the focal Rad51 DNA repair complex.

H. Rad51 DNA Repair Protein

Rad51, a eukaryotic homologue of the bacterial RecA protein involved in homologous recombination, catalyzes double-stranded break repair (DSBR) in damaged cells. Rad51 is highly overexpressed in tumor cells, and down-regulating its activity results in inhibition of double stranded break repair.

Cells defective for Rad51-mediated recombination show increased rates of mutagenesis and chromosomal rearrangements. The Rad51 protein plays a pivotal role in gene conversion during homologous recombination induced by ionizing (IR) or ultraviolet (UV) irradiation, DNA damaging agents, and replication elongation agents and is involved in sister-chromatid exchange (SCE).

Increased Rad51 mRNA and protein expression has been observed in malignant cells many times, in a variety of analyses including DNA microarray, RNA, and protein-based analyses (Maacke, Jost et al. 2000; Maacke, Opitz et al. 2000; Schwaibold, Detmar et al. 2000). In addition, it has been shown that down regulation of Rad51 expression levels in vivo in mice using antisense drug technology combined with radiation has prolonged survival significantly (Ohnishi, Taki et al. 1998), compared to control mice that died rapidly of their radioresistant brain tumors.

### SUMMARY OF THE INVENTION

Methods and compositions are provided for modulating cellular activity. In the subject methods, target cells are contacted with both a BER inhibitor, e.g. Ape1 inhibitor, a DSBR inhibitor, e.g., a Rad51 inhibitor, and a sensitizing agent, e.g., either a radiosensitizing agent and/or a chemotherapeutic agent. Also provided are pharmaceutical preparations, as well as kits thereof, that find use in practicing the subject methods. The subject methods find use in a variety of different applications, including the treatment of hosts suffering from cellular proliferative diseases, e.g., neoplastic diseases, viral diseases, premature aging deseases and degenerative diseases. The present invention provides a number of advantages. For example, the combination of both BER and DSBR inhibitor drugs with IPDR or IUDR radiosensitizer followed by radiation therapy inhibits both single and double strand break repairs (SSB and DSB, respectively) and thus increases the radiosensitivity and improves the efficacy of the treatments.

The present invention provides methods for modulating cellular activity based on the series of discoveries relating to the pivotal role that the BER and DSBR pathways play in a number of cellular functions, including those involved in disease states. Also provided are methods for inhibiting cell proliferation in an individual comprising administering to the individual a composition comprising a BER inhibitor, e.g. an Ape1 inhibitor, a DSBR inhibitor, e.g., a Rad51 inhibitor, and a sensitizing agent, e.g., either a radiosensitizing agent and/or a chemotherapeutic agent. Also provided herein is a method for inhibiting the growth of a cell comprising administering to said cell a composition comprising a BER inhibitor, e.g. an Ape1 inhibitor, a DSBR inhibitor, e.g., a Rad51 inhibitor, and a sensitizing agent, e.g., either a radiosensitizing agent (e.g. IPDR or IUDR) and/or a chemotherapeutic agent. Such methods can further include the step of providing radiation or DNA damaging agents after administration of said composition. In preferred embodiments the methods are performed in vivo and/or on cancerous cells and can be used with intra-operative treatments.

In another aspect, the present invention provides methods for inhibiting cell proliferation in an individual in vivo comprising administering to the individual a composition comprising a DSBR inhibitor, e.g., a Rad51 antisense molecule, and a BER inhibitor such as an Ape1 antisense molecule or methoxyamine. Also provided herein is a method for inhibiting the growth or killing of a cancerous cell or a viral infected cell comprising administering to said cell a composition comprising a DSBR inhibitor, e.g., a Rad51 antisense molecule and a BER inhibitor such as a Ape1 antisense molecule or methoxyamine.

In another aspect, provided herein is a method for inducing sensitivity to radiation and DNA damaging chemotherapeutics in an individual in vivo comprising administering to said individual a composition comprising a DSBR inhibitor, e.g., a Rad51 antisense molecule and a BER inhibitor such as a Ape1 antisense molecule or methoxyamine. Also provided herein is method for inducing sensitivity to radiation and DNA damaging chemotherapeutics in a cancerous cell comprising administering to said cell a composition comprising a DSBR inhibitor, e.g., a Rad51 antisense molecule and a BER inhibitor such as a Ape1 antisense molecule or methoxyamine. In one embodiment, the methods provided herein also include the step of administering radiation or DNA damaging agents to a cell.

Further provided herein, is an invention in which a DNA damaging agent and a DNA repair pathway inhibitor are combined into a single molecule, which is broken down in the body into its active components. Additionally, an invention is provided in which polymeric forms of the nucleoside analogue precursor, e.g. an oligonucleotide comprised of IPDR, are synthesized by a novel method. The resulting polymer can be administered in a number of different formulations, which are broken down in the body into monomeric components.

Further provided herein are kits for diagnosing and/or treating cancer comprising a BER inhibitor, a DSBR inhibitor, e.g., a Rad51 inhibitor, and a sensitizing agent, e.g., either a radiosensitizing agent and/or a chemotherapeutic agent. In one aspect, the kit is for adjunctive therapy for cancer. In a preferred embodiment, the kit comprises at least one of packaging, instructions, suitable buffers, controls, and pharmaceutically acceptable carriers.

In any or all of the above embodiments, the BER inhibitor, the DSBR inhibitor, and the sensitizing agent, e.g., either a radiosensitizing agent and/or a chemotherapeutic agent, or any combinations of them, can be administered either as a single formulation or as individual formulations administered in a sequential manner.

### BRIEF DESCRIPTION OF DRAWINGS

FIG. 1. Effect of Methoxyamine (MX) on cytotoxicity induced by IUDR in human A2780/cp70 (MMR-deficient) and A2780/cp70/chr3 (MMR-proficient) ovary carcinoma cell lines. Cells were treated for 48 hours with IUDR alone or with IUDR and 6 mM Methoxyamine. Surviving colonies were counted in triplicate 7-10 days after treatment. Error bars, Standard Error.

FIG. 2. Effect of Methoxyamine (MX) on cytotoxicity induced by IUDR in human HCT116 (MMR-deficient) and HCT116/3-6 (MMR-proficient) colon carcinoma cell lines. Cells were treated for 48 hours with IUDR alone (closed symbols) or with IUDR and 6 mM Methoxyamine (open symbols). Surviving colonies were counted in triplicate 7-10 days after treatment. Error bars, Standard Error.

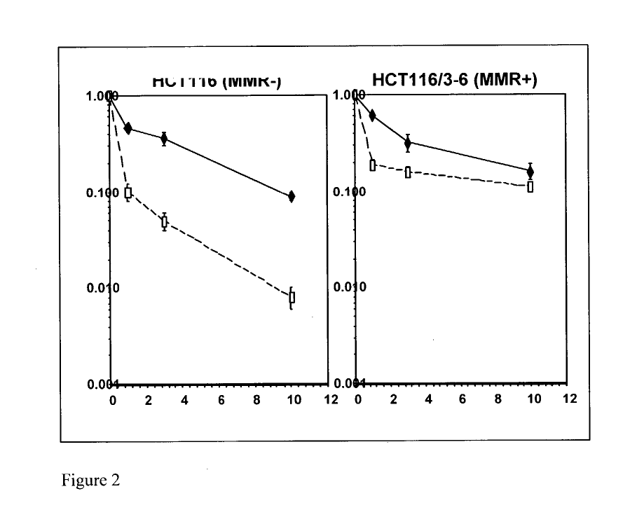

FIG. 3. Effect of Methoxyamine (MX) on cytotoxicity induced by FaraA in CHO cells proficient or deficient in Xrcc1 protein. Cells were treated for 24 hours with FaraA alone or with FaraA and 6 mM Methoxyamine. Surviving colonies were counted in triplicate 7-10 days after treatment and the experiment was repeated three times. Error bars, Standard Error.

FIG. 4. Effect of Methoxyamine (MX) on cytotoxicity induced by FaraA in human HCT116 (MMR-deficient) and HCT116/3-6 (MMR-proficient) colon carcinoma cell lines. Cells were treated for 24 hours with FaraA alone or with FaraA and 6 mM Methoxyamine. Surviving colonies were counted in triplicate 7-10 days after treatment. Error bars, Standard Error.

FIG. 5. Effect of Methoxyamine (MX) on cytotoxicity induced by FaraA in human A2780/cp70 (MMR-deficient) and A2780/cp70/chr3 (MMR-proficient) ovary carcinoma cell lines. Cells were treated for 48 hours with FaraA alone or with FaraA and 6 mM Methoxyamine. Surviving colonies were counted in triplicate 7-10 days after treatment. Error bars, Standard Error.

FIG. 6. Effect of Rad51 antisense on tumor growth delay induced by Doxorubicin on Human MDA-MB-231 Breast cancer cells grown as xenografts in Athymic Mice. The mice were treated i.p. with Rad51 antisense at 5 mg/kg on days 1 through 5, MX at 2 mg/kg on days 1 through 5, and with Doxorubicin at 1.5 mg/kg on day 4. The cycle was repeated three times with a two-day rest period between cycles.

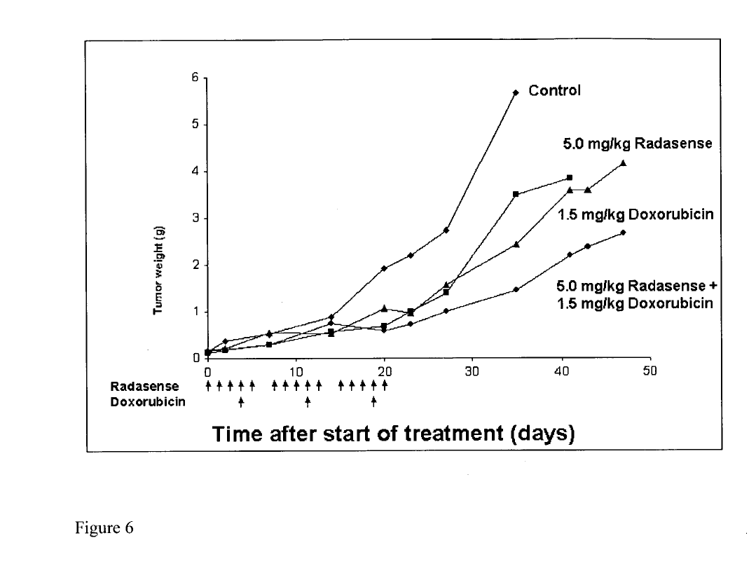

FIG. 7. The chemical structure of MX-IPDR

### DETAILED DESCRIPTION OF THE INVENTION

The present invention is based on the series of discoveries relating to the pivotal role that the BER and DSBR pathways play in a number of cellular functions, including those involved in disease states. In particular, the present invention is based in part on inhibiting the Ape1 and Rad51 proteins, which are key members of the BER and DSBR pathways, respectively. In the subject methods, target cells are contacted with both a BER inhibitor, e.g. a Ape1 inhibitor, and a DSBR inhibitor, e.g., a Rad51 inhibitor, as well as a sensitizing agent, e.g., either a radiosensitizing agent and/or a chemotherapeutic agent, where the cells may optionally be also treated by radiation. Also provided are pharmaceutical preparations, as well as kits thereof, that find use in practicing the subject methods. The subject methods find use in a variety of different applications, including the treatment of hosts suffering from cellular proliferative diseases, e.g., neoplastic diseases.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the elements that are described in the publications which might be used in connection with the presently described invention.

A BER inhibitor as defined herein inhibits the repair of single strand breaks by the BER pathway by at least 20% and preferably by at least 95%. A DSBR inhibitor as defined herein inhibits the repair of double strand breaks by the DSBR pathway by at least 20% and preferably by at least 95%. A protein inhibitor as defined herein inhibits the expression or translation of a protein-encoding nucleic acid or the biological activity of a peptide by at least 20%, and most preferably by at least 95%.

By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of additional moieties such as labels, or to increase the stability and half-life of such molecules in physiological environments. In addition, mixtures of naturally occurring nucleic acids and analogs can be made. Alternatively, mixtures of different nucleic acid analogs, and mixtures of naturally occurring nucleic acids and analogs may be made. The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

The nucleic acids herein, including antisense nucleic acids, and further described above, are recombinant nucleic acids. A recombinant nucleic acid is distinguished from naturally occurring nucleic acid by at least one or more characteristics. For example, the nucleic acid may be isolated or purified away from some or all of the nucleic acids and compounds with which it is normally associated in its wild type host, and thus may be substantially pure. For example, an isolated nucleic acid is unaccompanied by at least some of the material with which it is normally associated in its natural state, preferably constituting at least about 0.5%, more preferably at least about 5% by weight of the total nucleic acid in a given sample. A substantially pure nucleic acid comprises at least about 75% by weight of the total nucleic acid, with at least about 80% being preferred, and at least about 90% being particularly preferred. Alternatively, the recombinant molecule could be made synthetically, i.e., by a polymerase chain reaction, and does not need to have been expressed to be formed. The definition includes the production of a nucleic acid from one organism in a different organism or host cell. The antisense molecules hybridize under normal intracellular conditions to the target nucleic acid to inhibit either Rad51 or Ape1 expression or translation. The target nucleic acid is either DNA or RNA. In one embodiment, the antisense molecules bind to regulatory sequences for Rad51 or Ape1. In one embodiment, the antisense molecules bind to 5′ or 3′ untranslated regions directly adjacent to the coding region. Preferably, the antisense molecules bind to the nucleic acid within 1000 nucleotides of the coding region, either upstream from the start or downstream from the stop codon. In a preferred embodiment, the antisense molecules bind within the coding region of the Rad51 molecule. In one embodiment, the antisense molecules are not directed to the structural gene; this embodiment is particularly preferred when the antisense molecule is not combined with another antisense molecule. It is understood that any of the antisense molecules can be combined.

In one embodiment combinations of antisense molecules are utilized. In one embodiment, at least antisense molecule is selected from the 3′ untranslated region.

By “siRNA” or grammatical equivalents herein means a short double stranded RNA molecule which could induce a response within a cell which would lead to degradation of RNA molecules which contain homologous sequences to the siRNA.

In one embodiment, DNA repair inhibitors include the use of siRNA targeted at a DNA repair protein, e.g. Rad51 or Ape1. In another embodiment, DNA repair inhibitors can include combinations of siRNA targeted at a DNA repair protein, e.g. Rad51 or Ape1, and a different type of DNA repair inhibitor.

In an embodiment provided herein, the invention provides methods of treating disease states requiring inhibition of cellular proliferation. In a preferred embodiment, the disease state requires inhibition of the expression, translation or the biological activity at least one protein from the BER or DSBR DNA repair pathways as described herein. As will be appreciated by those in the art, a disease state means either that an individual has the disease, or is at risk to develop the disease.

Disease states which can be treated by the methods and compositions provided herein include, but are not limited to hyper-proliferative disorders. More particular, the methods can be used to treat, but are not limited to treating, cancer (further discussed below), viral diseases, autoimmune disease, arthritis, diabetes, graft rejection, inflammatory bowel disease, proliferation induced after medical procedures, including, but not limited to, surgery, angioplasty, and the like. Thus, in one embodiment, the invention herein includes application to cells or individuals afflicted or impending affliction with any one of these disorders.

The compositions and methods provided herein are particularly deemed useful for the treatment of cancer including solid tumors such as skin, breast, brain, cervical carcinomas, testicular carcinomas, etc.. More particularly, cancers that may be treated by the compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastom, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma [embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above identified conditions.

The individual, or patient, is generally a human subject, although as will be appreciated by those in the art, the patient may be animal as well. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of patient. In a preferred embodiment, the individual requires inhibition of cell proliferation. More preferably, the individual has cancer or a hyperproliferative cell condition.

The compositions provided herein may be administered in a physiologically acceptable carrier to a host, as previously described. Preferred methods of administration include systemic or direct administration to a tumor cavity or cerebrospinal fluid (CSF).

In a preferred embodiment, these compositions can be administered to a cell or patient, as is outlined above and generally known in the art for gene therapy applications. In gene therapy applications, the antisense molecules are introduced into cells in order to achieve inhibition of Rad51 or Ape1. “Gene therapy” includes both conventional gene therapy where a lasting effect is achieved by a single treatment, and the administration of gene therapeutic agents, which involves the one time or repeated administration of a therapeutically effective DNA or RNA. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83, 4143-4146 [1986]). The oligonucleotides can be modified to enhance their uptake, e.g. by substituting their negatively charged phosphodiester groups by uncharged groups.

Dosages and desired drug concentrations of pharmaceutical compositions of the present invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

In one aspect, the BER and DSBR inhibitors herein induce sensitivity to DNA damaging agents and radiation. Induced sensitivity (also called sensitization or hypersensitivity) can be measured by the cells tolerance to radiation or DNA damaging agents. For example, sensitivity, which can be measured, i.e., by toxicity, occurs if it is increased by at least 20%, more preferably at least 40%, more preferably at least 60%, more preferably at least 80%, and most preferably by 100% to 200% or more.

In an embodiment herein, the methods comprising administering the BER and DSBR inhibitors provided herein further comprise administering a DNA damaging agent or radiation. For the purposes of the present application the term ionizing radiation shall mean all forms of radiation, including but not limited to alpha, beta and gamma radiation and ultra violet light, gamma knife, fractionated beam, intraoperative radiation treatment, brachytherapy, electron beam radiotherapy, radio-antibody and external beam radiotherapy, which are capable of directly or indirectly damaging the genetic material of a cell or virus. The term irradiation shall mean the exposure of a sample of interest to ionizing radiation, and term radiosensitive shall refer to cells or individuals, which display unusual adverse consequences after receiving moderate, or medically acceptable (i.e., nonlethal diagnostic or therapeutic doses), exposure to ionizing irradiation. Preferred DNA damaging agents may include, but are not limited to, nucleoside analogues, alkylating agents, topoisomerase inhibitors, plant alkaloids, antitumor antibiotics, platinum derivatives and bioreductive drugs.

Further provided herein, is an invention in which a DNA damaging agent and a DNA repair pathway inhibitor are combined into a single molecule, which is broken down in the body into its active components. In a preferred embodiment, the BER DNA repair pathway inhibitor methoxyamine (MX) is covalently coupled with 2-iodopyrimidinone-2′-deoxyribose (IPDR) to form a novel compound, termed MX-IPDR. This molecule is prepared by first forming an active carbonyl at the 3′ and 5′ hydroxyls of the IPDR molecule, followed by the reacting the carbonyl intermediate with methoxyamine to form the desired compound. An invention is also provided in which polymeric forms of MX-IPDR can be synthesized.

Additionally, an invention is provided in which polymeric forms of the nucleoside analogue precursor, e.g. an oligonucleotide comprised of IPDR or IUDR, are synthesized by a novel method. The resulting polymer can be administered in a number of different oral, injectible and other formulations, which are broken down in the body into monomeric components.

In one embodiment herein, the BER and DSBR inhibitors provided herein are administered to prolong the survival time of an individual suffering from a disease state requiring the inhibition of the proliferation of cells. In a preferred embodiment, the individual is further administered radiation or a DNA damaging agent.

The methods also find use in a variety of therapeutic applications in which it is desired to modulate the activity in a target cell or collection of cells, where the collection of cells may be a whole animal or portion thereof, e.g., tissue, organ, etc. As such, the target cell(s) may be a host animal or portion thereof, or may be a therapeutic cell (or cells) which is to be introduced into a multicellular organism, e.g., a cell employed in gene therapy. In such methods, an effective amount of an active agent that modulates cell activity, e.g., decreases or inhibits cell growth, as desired, is administered to the target cell or cells, e.g., by contacting the cells with the agent, by administering the agent to the animal, etc. By effective amount is meant a dosage sufficient to modulate cell activity in the target cell(s), as desired.

In the subject methods, the active agent(s) may be administered to the targeted cells using any convenient means capable of resulting in the desired modulating of cellular activity. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the agents can be achieved in various ways, including oral, buccal, rectal, parenteral, intraperitoneal, intradermal, transdermal, intracheal, etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host. The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Also provided are kits for use in practicing the subject methods. The subject kits at least include an effective amount of an active agent, or pharmaceutical preparation thereof, as described above. The various components of the kit may be present in separate containers or certain compatible components may be precombined into a single container, as desired.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g. a piece or pieces of paper on which the information is printed, in the packaging of the kit, in a package insert, etc. Yet another means would be a computer readable medium, e.g. diskette, CD, etc., on which the information has been recorded. Yet another means that may be present is a website address which may be used via the internet to access the information at a removed site. Any convenient means may be present in the kits.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are specifically incorporated by reference in their entirety.

### EXAMPLES

### Example 1

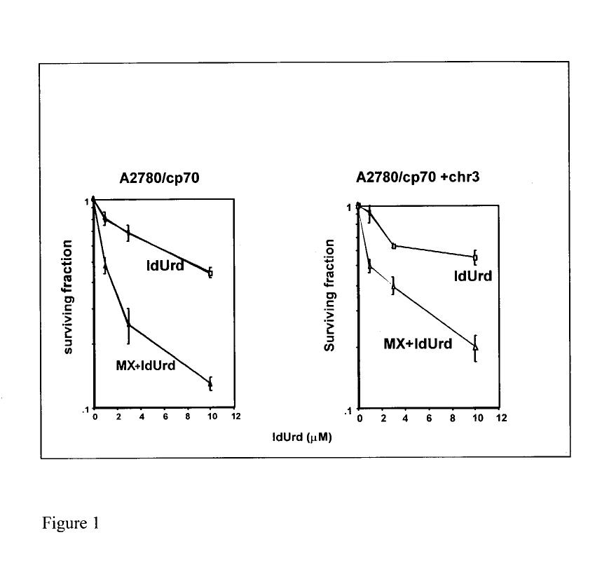

MX sensitizes human ovarian and colon cancer cells to IUDR and iodouridine-containing oligodeoxyribonucleotides (FIGS. 1 & 2). We treated cells with IUDR alone and with the combination MX+IUDR, two syngeneic human ovarian cancer cell lines characterized by different MMR status. The cisplatin-resistant derivative of the human ovarian cancer cell line A2780 (A2780/cp70) lacks MLH1 expression because of methylation of the hMLH1 gene promoter and their MMR positive subline (A2780/cp70+chr3) has hMLH1 reintroduced by chromosome 3 transfer.

For clonogenic survival studies in A2780/cp70 and A2780/cp70+chr3 cells, exponentially growing cells were similarly diluted and plated in complete RPMI 1640 medium (+400 μg/ml Hygromycin in A2780/cp70+chr3 cells). IUDR treatment (1, 3, 10 μM) in A2780/cp70 and A2780/cp70+chr3 cells was for 48 hours in RPMI 1640 (-Hygromycin) containing 10% dialyzed FBS with a replacement of the drug-containing medium after the initial 24 hours. IUDR-containing medium was then removed in cell populations, cultures were washed with PBS and incubated at 37° C. for 7-10 days in tissue culture medium containing 10% defined FBS (+dThd). Cell populations were treated simultaneously with IUDR and 6 mM Methoxyamine (MX) (Sigma, St Louis, Mo.) for 48 hours; plates were then washed with PBS and surviving colonies were counted after incubation at 37° C. in drug-free medium for 7-10 days. Drawing 1 shows that A2780/cp70 and cp70/ch3 cell lines were greatly sensitized to IUDR-induced cytotoxicity by treatment with 6 mM MX. The IC₅₀ for IUDR alone in A2780/cp70 cells was 8 μM whereas treatment with MX decreased the IC₅₀ for IUDR to 1 μM. The MMR positive subline A2780/cp70 chr3 was qualitatively similar to A2780/cp70 in its response to IUDR alone and to MX+IUDR.

The human colon cancer cell line HCT116 lacks MLH1 expression and their MMR positive subline (HCT116/chr 3-6) has hMLH1 reintroduced by chromosome 3 transfer. For clonogenic survival studies in HCT116 and HCT116/3-6 cells, exponentially growing cells were similarly diluted and plated in complete D-MEM medium (+400 μg/ml G418 in HCT116/3-6 cells). IUDR treatment (1, 3, 10 μM) in HCT116 and HCT116/3-6 cells was for 48 hours in D-MEM (-G418) containing 10% dialyzed FBS with a replacement of the drug-containing medium after the initial 24 hours. IUDR-containing medium was then removed in HCT116 cell populations, cultures were washed with PBS and incubated at 37° C. for 7-10 days in tissue culture medium containing 10% defined FBS (+dThd). HCT116 cell populations were treated simultaneously with IUDR and 6 mM Methoxyamine (MX) (Sigma, St Louis, Mo.) for 48 hours; plates were then washed with PBS and surviving colonies were counted after incubation at 37° C. in drug-free medium for 7-10 days. Drawing 2 shows that both cell lines were greatly sensitized to IUDR-induced cytotoxicity by treatment with 12 mM MX. The IC₅₀ for IUDR alone in HCT116 cells was 4.5 μM whereas treatment with MX decreased the IC₅₀ for IUDR to 0.5 μM. The MMR positive subline HCT116/chr3-6 was qualitatively similar to HCT116 in its response to IUDR alone and to MX+IUDR Thus, we present evidence that the MX-related BER inhibition is an effective approach to sensitize human tumors to the cytotoxic effects of IUDR.

### Example 2

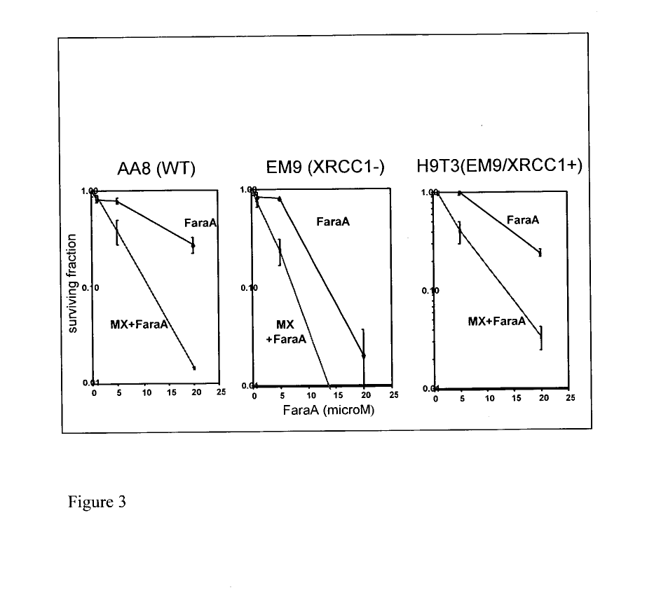

Methoxyamine (MX) increases sensitivity to Fludarabine (FaraA) in CHO cells (FIG. 3). AA8 and EM9 cells were obtained from the American Tissue Culture Collection (Manassas, Va.). H9T3 cells were a gift of Dr. L. H. Thompson (Lawrence Livermore National Laboratory, Livermore, Calif.). The parental CHO line clone AA8 was isolated as being heterozygous at the aprt locus; the mutant EM9 clone was isolated from AA8 cells following mutagenesis with EMS and carry a frameshift mutation in the XRCC1 gene resulting in a truncated polypeptide lacking two thirds of the normal sequence. Doubling times for AA8 and EM9 are 12 and 16 hours, respectively. H9T3-7-1 cells (referred in the text as H9T3) were derived from EM9 following transfection with a cosmid containing XRCC1 cDNA which corrects the DNA repair defect of EM9. H9T3 cells have a population doubling time of 15 hours.

We tested whether MX, a small molecule known to be BER inhibitor, could sensitize CHO cells to FaraA cytotoxicity. The wild type AA8 cells and the XRCC1 reconstituted H9T3 cells were all significantly sensitized to FaraA by treatment with 6 mM MX. The XRCC1 mutant EM9 cells were already hypersensitive to FaraA (IC₅₀ 7.5 microM) and could be further sensitized by MX. These data demonstrates that inhibitors of BER can be effective in sensitizing mammalian cells to FaraA.

### Example 3

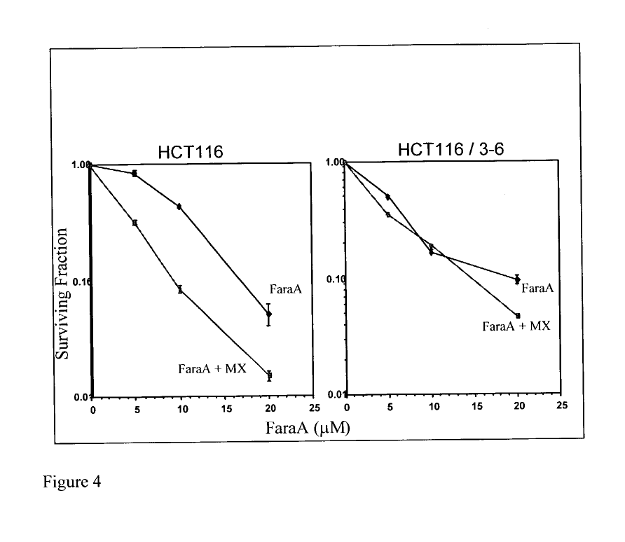

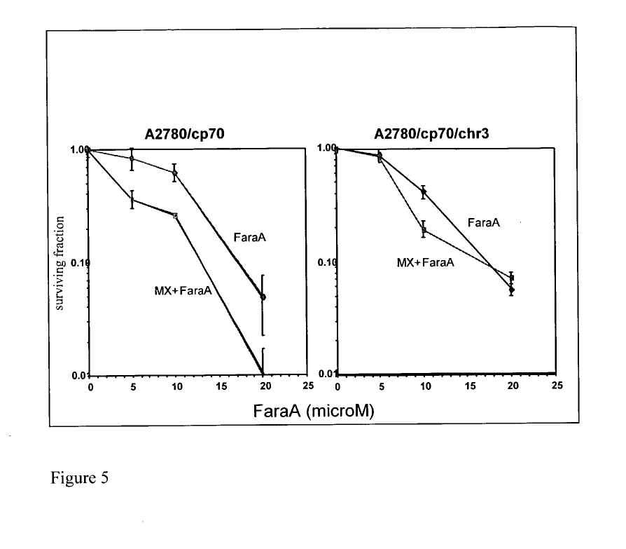

MX increases sensitivity to Fludarabine (FaraA) in human colon and ovary cancer cells (FIGS. 4 and 5). We tested whether MX could sensitize human cancer cells to FaraA cytotoxicity. The IC₅₀ for FaraA treatment in HCT116 cells was 9 μM whereas treatment with 6 mM MX decreased the IC₅₀ for FaraA to 3 μM. In the MMR positive subline HCT116/3-6 the IC₅₀ for FaraA was 5 μM and it was decreased to 3 μM in the presence of 6 mM MX (drawing 4).

The MMR-deficient human ovary cancer cells A2780/cp70 were also sensitized to FaraA by 6 mM MX. The IC₅₀ for FaraA alone was 11 μM, whereas treatment with 6 mM MX decreased the IC₅₀ to 4 μM (drawing 5). The MMR proficient subline A2780/cp70/ch3 was also decreased by the combination of MX and Fara.

### Example 5

### Effect of Doxorubicin, Rad51 Antisense and MX on Human MDA-MB-231 Breast Cells in Athymic Mice

Athymic mice are treated with Rad51 antisense alone, MX alone, and Rad51 antisense in combination with MX. All mice are also treated with Doxorubicin. Tumor fragment of about 2×2×2 mm derived from Human MDA-MB-231 breast cancer cells Athymic mice are implanted s.c. into the axilliary region of the mice. The mice are treated i.p. with Rad51 antisense at 5 mg/kg on days 1 through 5, MX at 2 mg/kg on days 1 through 5, and Doxorubicin at 1.5 mg/kg on day 4. The cycle is repeated three times with a two-day rest period between cycles. The animals are euthanatized 49 days after the beginning of the experiment, and the tumor size is measured by calipers. Reduction in tumor size and tumor cell killing are observed with either Rad51 antisense or MX treatments. A more significant effect is expected when both Rad51 antisense and MX are used simultaneously.

### Example 6

### Effect of IPDR, Rad51 Antisense, and MX on Human U87 MG Glioma Cells in Athymic Mice

Athymic mice are treated with Rad51 antisense alone, MX alone, and Rad51 antisense in combination with MX. All mice are also treated with IPDR. Tumor fragment of about 2×2×2 mm derived from Human U87 MG glioma cells are implanted s.c. into the axilliary region of the mice. The mice are treated i.p. with Rad51 antisense at 5 mg/kg on days 1 through 5, MX at 2 mg/kg on days 1 through 5, and IPDR at 250 mg/kg on day 4. The cycle is repeated three times with a two-day rest period between cycles. The animals are euthanatized 49 days after the beginning of the experiment, and the tumor size is measured by calipers. Reduction in tumor size and tumor cell killing are observed with either Rad51 antisense or MX treatments. A more significant effect is expected when both Rad51 antisense and MX are used simultaneously.

### Example 7

### Plasma Levels of IPDR in Athymic Mice

Athymic mice are treated with MX-IPDR or IPDR. Tumor fragment of about 2×2×2 mm derived from Human U87 MG Glioma Cells are implanted s.c. into the axilliary region of the mice. The mice are then treated ip with MX-IPDR at 250 mg/kg or IPDR at 250 mg/kg and samples taken at 0, 2, 5, 15, 45, 75, 120, 150, 210 and 350 min after treatment. The IPDR plasma levels are determined as previously reported by Kinsella et al. {Kinsella, 2000 #121}. The results establish an acceptable level of plasma IPDR serum levels required to act as a radiosensitizer.

### Example 8

### Plasma Levels of IUDR in Athymic Mice

Human colon cells (HCT 116) are treated with poly IUDR, an oligonucleotide comprised of IUDR, and IUDR. For clonogenic survival studies in HCT116, exponentially growing cells are similarly diluted and plated in complete D-MEM medium (+400 μg/ml G418 in HCT116/3-6 cells). Poly IUDR and IUDR treatment (1, 3, 10 μM) in HCT116 is for 48 hours in D-MEM (-G418) medium containing 10% dialyzed FBS, and the drug-containing medium is replaced after the initial 24 hours. Compound-containing medium is then removed from the HCT116 cell cultures, the cultures are washed with PBS, and incubated at 37° C. for 7-10 days in tissue culture medium containing 10% defined FBS (+dThd). Surviving colonies are counted after incubation at 37° C. in drug-free medium for 7-10 days. The cultures are greatly sensitized to both poly IUDR and IUDR-induced cytotoxicity by treatment.

### Example 9

### Human Clinical Trials with Binary IPDR/MX

Human clinical trials of IPDR/MX will be carried out in patients with a histological diagnosis of glioblastoma. No preselection for tumor sites and type of surgery will be required. Subjects will be given oral doses of IPDR/MX preferably at a dose of about 10 mg/m² every day for 42 days, but may be given the drug on a less frequent basis of every other day or every 7 days. Subjects will be administered standard radiation therapy and subjects will be followed to determine the safety and efficacy of the IPDR/MX.

### Example 10

### Solid-phase Synthesis of Iodouridine-containing Oligodeoxyribonucleotides

The iodouracil-containing oligodeoxyribonucleotides were synthesized by the solid-phase 2-cyanoethylphosphoramidite chemistry on an ABI 392-5 DNA synthesizer on 1 mmol scale, using the standard solid-phase 2-cyanoethylphosphoramidite program. Solutions in anhydrous acetonitrile containing 0.1 M 54-O-pixyl-5-iodouracil-24-deoxyriboside-34-O-(2-cyanoethyl-N,N-diisopropyl) phosphoramidite were used for the solid-phase couplings. When 54-O-pixyl-5-iodouracil-24-deoxyriboside-34-O-oxalyl-LCAA-CPG is used as the solid support for oligomer assembly, a pre-capping step is performed before initiating the solid-phase synthesis. Deblocking of the 54-pixyl group was effected by the use of 2.5% dichloroacetic acid in dichloromethane for the specified duration of time as determined by the synthesis programs. The deblocking fractions were collected and assayed for the released pixyl group to assess the step-wise coupling efficiency. Average coupling efficiencies were greater than 98%. Cleavage of the 5-iodouracil-containing oligomers from the oxalyl-CPG was achieved by a short treatment (10 min) of the support with 5% ammonium hydroxide in methanol at room temperature. Average yield of CPG cleavage was 98%. For the deprotection of the 5-iodouracil-containing oligodeoxyribonucleotides, a treatment of 70 min was used instead to fully deprotect the cyanoethyl groups on the phosphate. The supernatants obtained from the CPG cleavage and the oligomer deprotection reactions were evaporated to dryness under reduced pressure and the crude oligomers dissolved in 50 mM sodium phosphate buffer at pH 7. The crude oligomer solutions were then subjected to UV and reversed-phase HPLC analysis (Rainin Microsorb C18 4.6·250 mm, Woburn, Mass.) and purified by preparative reversed phase HPLC (Rainin Microsorb C18 10·250 mm). The columns were eluted with linear gradients of acetonitrile in 50 mM sodium phosphate, pH 7. Preparative HPLC fractions containing the pure full-length products were pooled, diluted with 50 mM sodium phosphate, pH 7, desalted on a C18 guard column, and eluted with 50% acetonitrile/water. The desalted oligomer solutions were diluted to 20% acetonitrile/water and stored in the freezer at −70° C. until use.

### Example 11

Solid-phase synthesis of iodopyrimidinone-containing oligodeoxyribonucleotides. The iodopyrimidinone-containing oligodeoxyribonucleotides are synthesized by the solid-phase 2-cyanoethylphosphoramidite chemistry on ABI 392-5 DNA synthesizer on 1 mmol scale, using the standard solid-phase 2-cyanoethylphosphoramidite program. Solutions in anhydrous acetonitrile containing 0.1 M 54-O-pixyl-5-iodo-2-pyrimidinone-24-deoxyriboside-34-O-(2-cyanoethyl-N,N-diisopropyl) phosphoramidite are used for the solid-phase couplings. When 54-O-pixyl-5-iodo-2-pyrimidinone-24-deoxyriboside-34-O-oxalyl-LCAA-CPG is used as the solid support for oligomer assembly, a pre-capping step is performed to the support before initiating the solid-phase synthesis. Deblocking of the 54-pixyl group is effected by the use of 2.5% dichloroacetic acid in dichloromethane for the specified duration of time as determined by the synthesis programs. The deblocking fractions are collected and assayed for the released pixyl group to assess the step-wise coupling efficiency. Average coupling efficiencies are greater than 98%. Cleavage of the 5-iodo-2-pyrimidinone-containing oligomers from the oxalyl- CPG is achieved by a short treatment (10 min) of the support with 5% ammonium hydroxide in methanol at room temperature. Average yield of CPG cleavage is 98%. For the deprotection of the 2-pyrimidinone-containing oligodeoxyribonucleotides, a treatment of 70 min is used instead to fully deprotect the cyanoethyl groups on the phosphate. The supernatants obtained from the CPG cleavage and the oligomer deprotection reactions are evaporated to dryness under reduced pressure and the crude oligomers dissolved in 50 mM sodium phosphate buffer at pH 7 (for the phosphodiester oligomers). The crude oligomer solutions are then subjected to UV and reversed-phase HPLC analysis (Rainin Microsorb C18 4.6·250 mm, Woburn, Mass.) and purified by preparative reversed phase HPLC (Rainin Microsorb C18 10·250 mm). The columns are eluted with linear gradients of acetonitrile in 50 mM sodium phosphate, pH 7. Preparative HPLC fractions containing the pure full-length products are pooled, diluted with 50 mM sodium phosphate, pH 7, and then desalted on a C18 guard column, eluting with 50% acetonitrile/ water. The desalted oligomer solutions are diluted to 20% acetonitrile/water and stored in the freezer at −70° C. until use.

### Example 12

### Synthesis of MX-IPDR

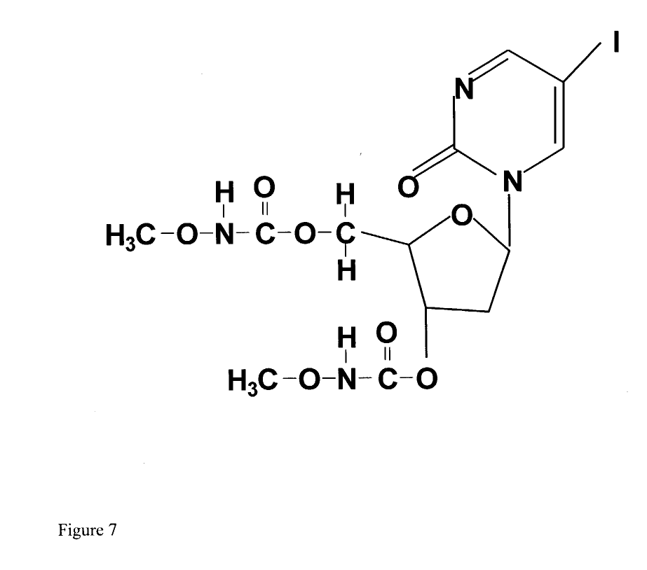

50 grams of IPDR are added to 300 ml of pyrimidine and 2 equivalents of carbonyl diimadazole are added to the mixture. The reaction is carried out at room temperature. 2 equivalents of methoxyamine are added directly to the reaction mixture and maintained at room temperature. The reaction mixture is then evaporated to dryness, water is added and the product crystallized. The purity of the resulting MX-IPDR is determined by NMR, mass spec and by CHN analysis. The structure of MX-IPDR is shown in FIG. 7.

### REFERENCE LIST

Bhattacharyya, A., U. S. Ear, et al. (2000). “The Breast Cancer Susceptibility Gene BRCA1 Is Required for Subnuclear Assembly of Rad51 and Survival following Treatment with the DNA Cross-linking Agent Cisplatin.” J Biol Chem 275(31): 23899-23903.

Bogliolo, M., R. M. Taylor, et al. (2000). “Reduced ligation during DNA base excision repair supported by BRCA2 mutant cells.” Oncogene 19(50): 5781-7.

Brandon, M. L., L. Mi, et al. (2000). “5-chloro-2′-deoxyuridine cytotoxicity results from base excision repair of uracil subsequent to thymidylate synthase inhibition.” Mutat Res 459(2): 161-9.

Caldecott, K. W. (2001). “Mammalian DNA single-strand break repair: an X-ra(y)ted affair.” Bioessays 23(5): 447-55.

Chang, C. N., S. L. Doong, et al. (1992). “Conversion of 5-iodo-2-pyrimidinone-2′-deoxyribose to 5-iodo-deoxyuridine by aldehyde oxidase: Implication in hepatotropic drug.” Biochemical Pharmacology 43(10): 2041-2298.

Chaudhry, M. A. and M. Weinfeld (1997). “Reactivity of human apurinic/apyrimidinic endonuclease and Escherichia coli exonuclease III with bistranded abasic sites in DNA.” J Biol Chem 272(25): 15650-5.

Chen, J. J., D. Silver, et al. (1999). “BRCA1, BRCA2, and Rad51 operate in a common DNA damage response pathway.” Cancer Research 59((7 Suppl S)): 1752S-1756S.

Dillehay, L. E., L. H. Thompson, et al. (1984). “DNA-strand breaks associated with halogenated pyrimidine incorporation.” Mutat Res 131(3-4): 129-36.

Fornace, A. J., Jr., P. P. Dobson, et al. (1990). “Enhancement of radiation damage in cellular DNA following unifilar substitution with iododeoxyuridine.” Int J Radiat Oncol Biol Phys 18(4): 873-8.

Harrison, L., Z. Hatahet, et al. (1998). “Multiple damaged sites in DNA: interactions with Escherichia coli endonucleases III and VIII.” Nucleic Acids Res 26(4): 932-41.

Horton, J. K., A. Baker, et al. (2002). “Involvement of DNA polymerase in protection against the cytotoxicity of oxidative DNA damage.” DNA Repair 1(4): 317-333.

Keating, M. J., E. Estey, et al. (1985). “Evolution of clinical studies with high-dose cytosine arabinoside at the M. D. Anderson Hospital.” Semin Oncol 12(2 Suppl 3): 98-104.

Keating, M. J., H. Kantarjian, et al. (1989). “Fludarabine: a new agent with major activity against chronic lymphocytic leukemia.” Blood 74(1): 19-25.

Kim, K. J., I. Chakrabarty, et al. (2002). “Modulation of Base Excision Repair Alters Cellular Sensitivity to UVA1 but not to UVB.” Photochemistry and Photobiology 75(5): 507-512.

Kinsella, T. J. (1996). “An Approach to the Radiosensitization of Human Tumors.” Cancer J Sci Am 2(4): 184.

Kinsella, T. J., P. P. Dobson, et al. (1987). “Enhancement of X ray induced DNA damage by pre-treatment with halogenated pyrimidine analogs.” Int J Radiat Oncol Biol Phys 13(5): 733-9.

Kinsella, T. J., J. E. Schupp, et al. (2000). “Preclinical Study of the Systemic Toxicity and Pharmacokinetics of 5-Iodo-2-deoxypyrimidinone-2′-deoxyribose as a Radiosensitizing Prodrug in Two, Non-Rodent Animal Species: Implications for Phase I Study Design.” Clin Cancer Res 6(9): 3670-3679.

Kinsella, T. J., K. A. Vielhuber, et al. (2000). “Preclinical Toxicity and Efficacy Study of a 14-day Schedule of Oral 5-Iodo-2-pyrimidinone-2′-deoxyribose as a Prodrug for 5-Iodo-2′-deoxyuridine Radiosensitization in U251 Human Glioblastoma Xenografts.” Clin Cancer Res 6(4): 1468-1475.

Lindahl, T. (1993). “Instability and decay of the primary structure of DNA.” Nature 362(6422): 709-15.

Liu, L., Y. Nakatsuru, et al. (2002). “Base Excision Repair as a Therapeutic Target in Colon Cancer.” Clinical Cancer Research 8: 2985-2991.

Liu, L., P. Taverna, et al. (1999). “Pharmacologic disruption of base excision repair sensitizes mismatch repair-deficient and -proficient colon cancer cells to methylating agents.” Clin Cancer Res 5(10): 2908-17.

Maacke, H., K. Jost, et al. (2000). “DNA repair and recombination factor Rad51 is overexpressed in human pancreatic adenocarcinoma.” Oncogene 19(23): 2791-2795.

Maacke, H., S. Opitz, et al. (2000). “Over-expression of wild-type Rad51 correlates with histological grading of invasive ductal breast cancer.” International Journal of Cancer 88(6): 907-913.

Miller, E. M., J. F. Fowler, et al. (1992). “Linear-quadratic analysis of radiosensitization by halogenated pyrimidines. I. Radiosensitization of human colon cancer cells by iododeoxyuridine.” Radiat Res 131(1): 81-9.

Modesti, M. and R. Kanaar (2001). “DNA repair: spot(light)s on chromatin.” Current Biology 11: R229-232.

Neddermann, P. and J. Jiricny (1994). “Efficient Removal of Uracil from G-U Mispairs by the Mismatch- Specific Thymine DNA Glycosylase from HeLa Cells.” Proceedings of the National Academy of Sciences 91: 1642-1646.

Ohnishi, T., T. Taki, et al. (1998). “In Vitroandin VivoPotentiation of Radiosensitivity of Malignant Gliomas by Antisense Inhibition of the RAD51 Gene.” Biochemical and Biophysical Research Communications 245(2): 319-324.

Petronzelli, F., A. Riccio, et al. (2000). “Biphasic Kinetics of the Human DNA Repair Protein MED1 (MBD4), a Mismatch-specific DNA N-Glycosylase.” J. Biol. Chem 275: 32422-32429.

Pierce, A. J., J. M. Stark, et al. (2001). “Double-strand breaks and tumorigenesis.” Trends in Cell Biology 11(11): S52-59.

Schwaibold, H., P. Detmar, et al. (2000). “Over-expression of wild-type Rad51 correlates with histological grading of transitional cell carcinoma of the bladder.” Journal of Urology 165(5 Supplement): 109.

Storniolo, A. M., N. H. Enas, et al. (1999). “An investigational new drug treatment program for patients with gemcitabine: results for over 3000 patients with pancreatic carcinoma.” Cancer 85(6): 1261-8.

Taverna, P., H. S. Hwang, et al. (2003). “Inhibition of Base Excision Repair Potentiates Iododeoxyuridine-induced Cytotoxicity and Radiosensitization.” Cancer Res 63(4): 838-846.

Taverna, P., L. Liu, et al. (2001). “Methoxyamine potentiates DNA single strand breaks and double strand breaks induced by temozolomide in colon cancer cells.” Mutat Res 485: 269-281.

Tomicic, M. T., R. Thust, et al. (2001). “DNA Polymerase β Mediates Protection of Mammalian Cells against Ganciclovir-induced Cytotoxicity and DNA Breakage.” Cancer Research 61(20): 7399-7403.

Wilson, S. H. (1998). “Mammalian base excision repair and DNA polymerase beta.” Mutat Res 407(3): 203-15.

Wilstermann, A. M. and N. Osheroff (2001). “Base Excision Repair Intermediates as Topoisomerase II Poisons.” J. Biol. Chem 276: 46290-46296.

### PATENT LIST

Gerson, S. L. and L. Liu (2002). Methoxyamine potentiation of temozolomide anti-cancer activity. USA, Case Western Reserve University. U.S. Pat. No. 6,465,448.

Gerson, S. L. and L. i. Liu (2002). Methoxyamine combinations in the treatment of cancer. USA, Case Western Reserve University. U.S. patent application Ser. No. 20020198264.

Kelley, M. R. (2002). Methods and compositions for the use of apurinic/apyrimidinic endonucleases. USA, Advanced Research And Technology Institute, Indianapolis, Ind. U.S. Pat. No. 6,406,917.

Kelley, M. R., J. Duguid, et al. (1999). Methods and compositions for the use of apurinic/apyrimidinic endonucleases. USA, Advanced Research & Technology Institute. U.S. Pat. No. 5,919,643.

Kelley, M. R., J. Duquid, et al. (2001). Methods and compositions for the use of apurinic/apyrimidinic endonucleases. USA, Advanced Research & Technology Institute, Bloomington, Ind. U.S. Pat. No. 6,190,661.

Vallerga, A., H. Zeng, et al. (2000). Novel antisense inhibition of Rad51. PCT. International, Pangene corporation. WO 00/47231.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention

What is claimed:

1. A method of modulating activity of a cell, said method comprising:
contacting said cell with:
a) a DSBR inhibitor;
b) a BER inhibitor; and
c) a sensitizer agent.

2. The method according to claim 1, wherein said DSBR inhibitor inhibits Rad51.

3. The method according to claim 2, wherein said Rad51 inhibitor is a small molecule.

4. The method according to claim 2, wherein said Rad51 inhibitor is an antisense molecule.

5. The method according to claim 3, wherein said Rad51 inhibitor is an antisense oligonucleotide molecule.

6. The method according to claim 2, wherein said RaD51 inhibitor is a siRNA molecule.

7. The method according to claim 2, wherein said Rad51 inhibitor is a peptide inhibitor.

8. The method according to claim 2, wherein said Rad51 inhibitor is a small molecule chemical entity.

9. The method according to claim 8, wherein said Rad51 inhibitor is a p53 polypeptide or p53 oligopeptide.

10. The method according to claim 2, wherein said Rad51 inhibitor is a modified nucleotide, nucleoside or base.

11. The method according to claim 1, wherein said sensitizing agent is a radiosensitizing agent.

12. The method according to claim 11, wherein said method further comprises contacting said cell with radiation therapy.

13. The method according to claim 11, wherein said radiosensitizing agent comprises a halogenated pyrimidine.

14. The method according to claim 11, wherein said radiosensitizing agent comprises a halogenated purine.

15. The method according to claim 13, wherein said halogenated pyrimidine comprises a thymidine analogue.

16. The method according to claim 15, wherein said thymidine analogue comprises 5-iodo-2-deoxy-uridine (IUDR) or 5-brome-2-deoxy-uridine (BUDR) or 5-chloro-2-deoxyuridine (CUDR) or 5-fluoro-2-deoxy-uridine (FUDR).

17. The method according to claim 15, wherein said thymidine analogue comprises a radiolabelled halogen.

18. A method according to claim 13, wherein the radiosentizing agent comprises a 5-iodo-2-pyrimidinone deoxyribose (IPDR) or 5-bromo-2-pyrimidinone deoxyribose (BPDR) or 5-chloro-2-pyrimidinone deoxyribose (CPDR) or 5-fluoro-2-pyrimidinone deoxyribose (FPDR).

19. The method according to claim 13, wherein said halogenated pyrimidine contains a radiolabelled halogen.

20. The method according to claim 11, wherein the radiosentizing agent comprises a multi-functional compound comprised of an antibody that binds to a receptor on said cell and with the antibody containing a radioactive atom.

21. The method according to claim 1, wherein said BER inhibitor inhibits Ape1.

22. The method according to claim 1, wherein said BER inhibitor is a small molecule.

23. The method according to claim 1, wherein said BER inhibitor is an antisense molecule.

24. The method according to claim 1, wherein said BER inhibitor is an antisense oligonucleotide molecule.

25. The method according to claim 1, wherein said BER inhibitor is an SiRNA or RNAi molecule.

26. The method according to claim 1, wherein said BER inhibitor is an E3330-like compound.

27. The method according to claim 1, where the BER inhibitor is an alkoxyamine.

28. The method according to claim 28, wherein said alkoxyamine inhibitor comprises methoxyamine (MX) or derivatives thereof.

29. The method of claim 1, wherein said sensitizing agent is a chemotherapeutic agent.

30. The method according to claim 29, wherein said chemotherapeutic drug is a topoisomerase inhibitor.

31. The method according to claim 30, wherein said topoisomerase inhibitor is selected from the following group: etoposide, teniposide, camptothecin, captothecin 10-hydroxy, irinotecan, topotecan, lucanthone.

32. The method according to claim 29, wherein said chemotherapeutic drug is an alkylating agent.

33. The method according to claim 32, wherein said alkylating agent is selected from the following group: dacarbazine, streptozotocin, procarbazine, carmustine, semustine, lomustine, sarmustine, fotemustine, busulphan, treosulphan, mechloretamine, cyclophosphamide, iphosphamide, chlorambucil, melphalan, hexamethylmelamine.

34. The method according to claim 29, wherein said chemotherapeutic drug comprises a nucleoside analogue.

35. The method according to claim 34, wherein said nucleoside analogue is selected from the following group: 5-azacytidine, cytosine arabinoside, fludarabine, iododeoxyuridine, bromodeoxyuridine, chlorodeoxyuridine, fluorodeoxyuridine, gemcitabine.

36. The method according to claim 29, wherein said chemotherapeutic drug comprises a plant alkaloid.

37. The method according to claim 36, wherein said plant alkaloid is selected from the following group: vinblastine, vincristine, vindesine.

38. The method according to claim 29, wherein said chemotherapeutic drug comprises an antitumor antibiotic.

39. The method according to claim 38, wherein said antitumor antibiotic is selected from the following group: doxorubicin, daunorubicin, actinomycin, bleomycin, mytomycin, mytramycin, elsamitrucin, mitoxantrone.

40. The method according to claim 29, wherein said chemotherapeutic drug comprises a platinum derivative.

41. The method according to claim 40, wherein said platinum derivative is selected from the following group: cisplatin, carboplatin, oxaliplatin, satraplatin.

42. The method according to claim 29, wherein said chemotherapeutic drug comprises a bioreductive drug.

43. The method according to claim 42, wherein said bioreductive drug is selected from the following group: porfiromycin, AQ4N, Tirapazamine, EO9 (Neoquin).

44. The method according to claim 1, wherein said sensitizing agent is an oligonucleotide comprised of halogenated pyrimidinones.

45. The method according to claim 1, wherein said sensitizing agent is an oligonucleotide comprised of halogenated pyrimidines.

46. The method according to claim 1, wherein said inhibitor is a compound containing a BER inhibitor and DNA damaging agent.

47. The method according to claim 1, wherein said inhibitor is a compound containing a DSBR inhibitor and DNA damaging agent.

48. The method according to claim 1, wherein said modulating comprises at least inhibiting cell growth.

49. The method according to claim 48, wherein said at least inhibiting cell growth comprising killing said cell.

50. The method according to claim 1, wherein said cell is present in a living organism.

51. The method according to claim 50, wherein said contacting comprises:
administering an effective amount of said BER inhibitor, DSBR inhibitor, and said sensitizing agent to said organism.

52. The method according to claim 51, wherein said method is a method of treating said living organism for a cellular proliferative disease.

53. The method according to claim 52, wherein said tumor cells are selected from the group consisting of brain, lung, liver, spleen, kidney, lymph node, small intestine, pancreas, blood cells, colon, stomach, endometrium, prostate, testicle, ovary, cervix, skin, head and neck, esophagus, bone marrow and blood tumor cells.

54. The method according to claim 51, wherein said method is a method of treating said living organism for a viral disease.

55. The method according to claim 51, wherein said method is a method of treating said living organism for a degenerative diseases.

56. The method according to claim 1, wherein the DSBR, BER and sensitizing agent are administered sequentially.

57. A compound which is an oligonucleotide comprising halogenated pyrimidinones.

58. The compound according to claim 57, where the halogenated Pyrimidinone is a 5-iodo-2-pyrimidinone deoxyribose (IPDR) or 5-bromo-2-pyrimidinone deoxyribose (BPDR) or 5-chloro-2-pyrimidinone deoxyribose (CPDR).

59. The compound according to claim 58, where the number of pyrimidinone monomers is between two and twenty.

60. A compound which is an oligonucleotide comprising halogenated pyrimidines.

61. The compound according to claim 60, where the halogenated pyrimidine is a thymidine analogue.

62. The compound according to claim 60, where the halogenated pyrimidine is a cytidine analogue.

63. The compound according to claim 60, where the halogenated pyrimidine is 5-iodo-2-deoxy-uridine (IUDR) or 5-bromo-2-deoxy-uridine (BUDR) or 5-chloro-2-deoxy-uridine (CUDR).

64. The compound according to claim 60, where the number of halogenated pyrimidines monomers is between two and twenty.

65. A multi-functional compound comprising an inhibitor of DNA repair and a DNA damaging compound.

66. The compound according to claim 65, where the DNA damaging compound is an alkylating agent, topoisomerase inhibitor, platinum drug, plant alkaloid, bioreductive drug, and antitumor antibiotic.

67. The compound according to claim 65, where the DNA repair inhibitor is a BER inhibitor and a DSBR inhibitor.

68. The compound according to claim 65, where the DNA repair inhibitor is a BER inhibitor or a RAD51 inhibitor.

72. The compound according to claim 65, wherein the DNA damaging agent is a halogenated pyrimidinone and the DNA repair inhibitor is MX.

73. The compound according to claim 65, where in the DNA damaging agent is a halogenated pyrimidinone and the DNA repair inhibitor is an alkoxyamine.

74. The compound according to claim 65, wherein the DNA damaging agent is IPDR and the DNA repair inhibitor is MX.

75. A multi-functional compound comprising an inhibitor of DNA repair and a nucleoside analogue DNA.

76. The compound according to claim 75, where the base of the nucleoside analogue is a halogenated pyrimidine or halogenated purine.

77. The compound according to claim 75, where the base of the nucleoside analogue is a halogenated pyrimidinone or a halogenated purinone.

78. A compound which is an oligonucleotide comprising MX-IPDR.

79. A multi-functional compound comprising an antibody that binds to a receptor on said cell and with said antibody containing a radioactive atom.

80. The composition according to claim 79, where the radioactive element is selected from the group consisting of iodine, yttrium, technetium, indium and rhenium.

81. The method according to claim 12, wherein said radiation therapy consists of a gamma knife, fractionated beam, intraoperative radiation treatment, brachytherapy, electron beam radiotherapy, radioantibody and/or external beam radiotherapy.

82. A method of modulating activity of a cell, said method comprising:
contacting said cell with:
a) a BER inhibitor; and
b) a sensitizer agent.

83. The method of claim 82, wherein said sensitizing agent is a chemotherapeutic agent.

84. The method according to claim 83, wherein said chemotherapeutic drug is a topoisomerase inhibitor.

85. The method according to claim 84, wherein said topoisomerase inhibitor is selected from the following group: etoposide, teniposide, camptothecin, captothecin 10-hydroxy, irinotecan, topotecan, lucanthone.

86. The method according to claim 83, wherein said chemotherapeutic drug is an alkylating agent.

87. The method according to claim 86, wherein said alkylating agent is selected from the following group: dacarbazine, streptozotocin, procarbazine, semustine, lomustine, fotemustine, busulphan, treosulphan, mechloretamine, cyclophosphamide, iphosphamide, chlorambucil, melphalan, hexamethylmelamine.

88. The method according to claim 83, wherein said chemotherapeutic drug comprises a nucleoside analogue.

89. The method according to claim 88, wherein said nucleoside analogue is selected from the following group: 5-azacytidine, cytosine arabinoside, fludarabine, iododeoxyuridine, bromodeoxyuridine, fluorodeoxyuridine, gemcitabine.

90. The method according to claim 83, wherein said chemotherapeutic drug comprises a plant alkaloid.

91. The method according to claim 90, wherein said plant alkaloid is selected from the following group: vinblastine, vincristine, vindesine.

92. The method according to claim 83, wherein said chemotherapeutic drug comprises an antitumor antibiotic.

93. The method according to claim 92, wherein said antitumor antibiotic is selected from the following group: doxorubicin, daunorubicin, actinomycin, bleomycin, mytomycin, mytramycin, elsamitrucin, mitoxantrone.

94. The method according to claim 83, wherein said chemotherapeutic drug comprises a platinum derivative.

95. The method according to claim 94, wherein said platinum derivative is selected from the following group: cisplatin, carboplatin, oxaliplatin, satraplatin.

96. The method according to claim 83, wherein said chemotherapeutic drug comprises a bioreductive drug.

97. The method according to claim 96, wherein said bioreductive drug is selected from the following group: porfiromycin, AQ4N, Tirapazamine, EO9 (Neoquin).

98. The method according to claim 82, wherein said modulating comprises at least inhibiting cell growth.

99. The method according to claim 98, wherein said at least inhibiting cell growth comprising killing said cell.

100. The method according to claim 99, wherein said cell is present in a living organism.

101. The method according to claim 100, wherein said contacting comprises administering an effective amount of said BER inhibitor and said sensitizing agent to said organism.

102. The method according to claim 101, wherein said method is a method of treating said living organism for a cellular proliferative disease.

103. The method according to claim 101, wherein said method is a method of treating said living organism for a viral disease.

104. The method according to claim 101, wherein said method is a method of treating said living organism for a degenerative diseases.

105. The method according to claim 101, wherein said tumor cells are selected from the group consisting of brain, lung, liver, spleen, kidney, lymph node, small intestine, pancreas, blood cells, colon, stomach, endometrium, prostate, testicle, ovary, cervix, skin, head and neck, esophagus, bone marrow and blood tumor cells.

106. The method according to claim 100, wherein the BER and sensitizing agent are administered sequentially.

107. A pharmaceutical formulation comprising a BER inhibitor, a DSBR inhibitor and sensitizing agent.

108. A pharmaceutical formulation comprising a BER inhibitor, Rad51 inhibitor and sensitizing agent.

109. A pharmaceutical formulation comprising a Ape1 inhibitor, a Rad51 inhibitor and sensitizing agent.

110. A pharmaceutical formulation comprising a chemotherapeutic drug, a BER inhibitor and a Rad51 inhibitor.

111. A pharmaceutical formulation comprising an oligonucleotide comprised of halogenated pyrimidinones and radiation therapy.

112. A pharmaceutical formulation comprising an oligonucleotide comprised of halogenated pyrimidines and radiation therapy.

113. A pharmaceutical formulation comprising a multi-functional compound comprised of an inhibitor of DNA repair and a DNA damaging compound.

114. The composition of any of claim 113, which contains a pharmaceutically acceptable dosage of the multi-functional compound which ranges from about 0.001 g/m² to about 50 g/m² of human body weight.
